(19) United States
(12) Patent Application Publication
(10) Pub. No.: US 2026/0108651 A1
(43) Pub. Date: Apr. 23, 2026

(54) AROMA DIFFUSER

(71) Applicant: Aurora International Limited, Hong Kong (CN)

(72) Inventors: Chi Ko Jeckle Chan, Hong Kong (CN); Shuaisi Ye, Shenzhen (CN)

(21) Appl. No.: 19/367,678

(22) Filed: Oct. 23, 2025

(30) Foreign Application Priority Data
Oct. 23, 2024 (CN) 202422568760.4

(51) Int. Cl. A61L 9/03

(52) U.S. Cl. CPC A61L 9/032; A61L 9/037; A61L 2209/133; A61L 2209/135

(57) ABSTRACT

An aroma diffuser having a base including a cavity; a containment receptacle contained in the cavity; and a control motherboard arranged inside the cavity of the base below the containment receptacle. The aroma diffuser further having an essential oil storage cartridge that includes a partition separating a chamber in the essential oil storage cartridge into an air intake chamber and an essential oil storage chamber storing essential oil; an electric heating assembly that is in thermal connectivity with the essential oil storage chamber; and a plurality of contacts, mounted on an exterior surface of the essential oil storage cartridge, coupled to the electric heating assembly for electrically connecting the electric heating assembly to the control motherboard. The essential oil storage cartridge is removably inserted into the containment receptacle of the base.

### CROSS-REFERENCE TO RELATED APPLICATIONS

This application claims priority to and the benefit of foreign patent application no. 202422568760.4 filed at the China National Intellectual Property Administration on Oct. 23, 2024, the entire content of which is incorporated herein by reference.

### BACKGROUND

### Field

The present disclosure generally relates to aroma diffusers, and more particularly, to an aroma diffuser using an electric heating element atomizer.

### BACKGROUND

Aroma diffusers are devices that decompose water molecules and dissolved plant essential oils into a nano-scale cold mist with molecules of an approximate diameter of 0.15 microns and emits the mist into the surrounding air, filling the air with fragrance. They are suitable for various places such as homes, hotel rooms, lobbies, aisles, guest rooms, etc.

At present, aroma diffusers of essential oils mainly use the following dispersion (atomization) methods:
1. Ultrasonic ceramic sheet vibration: most of these types of machines need added water to operate. However, once the water dries out, scorching easily leads to damage of the ultrasonic ceramic sheet. Even though, when working, the machines will change the humidity of the room, because of the need to use water the machine usually needs to have a water cavity. Thus, the machine cannot be placed in different orientations to avoid leaking of water from the water cavity.
2. Gas injection: these types of machines need to use high-pressure gas tanks, some models using air pumps to work. However, because the mixing chamber cannot be replaced in this type of aroma diffuser, it is easy to result in the mixing of different odors. Also, these types of machines are loud when working;
3. Fans: these types of machines use gas mixed with essential oils for dissipation, but the emission speed is relatively slow when the room temperature is relatively low.

In view of the above, an aroma diffuser is a desirable having a low-profile threshold that requires minimal transportation/shipping space and, at the same time, high assembly efficiency.

### SUMMARY

The following description presents a simplified summary of one or more aspects of a disclosed aroma diffuser using an electric heating element atomizer to provide a basic understanding of such aspects. This summary is not an extensive overview of all contemplated features of the disclosure and is intended neither to identify key or critical elements of all aspects of the disclosure nor to delineate the scope of any or all aspects of the disclosure. Instead, its sole purpose is to present some concepts of one or more aspects of the disclosure in a simplified form as a prelude to the more detailed description that is presented later herein.

According to various aspects, the subject innovation relates to providing an aroma diffuser using an electric heating element atomizer.

In one aspect, the disclosure provides an aroma diffuser having a base including a cavity; a containment receptacle contained in the cavity; and a control motherboard is arranged inside the cavity of the base below the containment receptacle. The aroma diffuser further having an essential oil storage cartridge that includes a partition separating a chamber in the essential oil storage cartridge into an air intake chamber and an essential oil storage chamber storing essential oil; an electric heating assembly that is in thermal connectivity with the essential oil storage chamber; and a plurality of contacts, mounted on an exterior surface of the essential oil storage cartridge, coupled to the electric heating assembly for electrically connecting the electric heating assembly to the control motherboard. The essential oil storage cartridge is removably inserted into the containment receptacle of the base.

Another aspect of the disclosure provides that the essential oil storage cartridge further having an integrated circuit including information about the essential oil in the essential oil storage chamber; where the integrated circuit is coupled to the plurality of contacts of the essential oil storage cartridge; and where the control motherboard can determine how to heat the essential oil using the information from the integrated circuit.

Another aspect of the disclosure provides that the information contained in the integrated circuit for the essential oil further includes information regarding the electric heating assembly in the essential oil storage cartridge.

Another aspect of the disclosure provides that the essential oil storage cartridge further having an arrangement of magnets on the exterior surface of the essential oil storage cartridge that aligns the plurality of contacts of the essential oil storage cartridge to electrically connect to a matching set of contacts on the control motherboard when the essential oil storage cartridge is inserted into the containment receptacle of the base.

Another aspect of the disclosure provides the electric heating assembly is embedded into a bottom of the essential oil storage cartridge, and when the essential oil storage cartridge is inserted into the containment receptacle of the base, the electric heating assembly is electrically connected to the control motherboard to allow the control motherboard to control the electric heating assembly so as to heat and atomize the essential oil contained in the essential oil storage chamber.

Another aspect of the disclosure provides the partition allows the essential oil storage chamber and the air intake chamber to be connected at a top portion of the partition, and the essential oil storage chamber is filled with an absorbent material infused with the essential oil.

Another aspect of the disclosure provides the electric heating assembly includes a heating member and a plurality of pins electrically connected to the heating member; wherein the control motherboard comprises conductive contacts; and wherein the containment receptacle is provided with openings on a bottom surface such that when the essential oil storage cartridge is inserted into the containment receptacle, the plurality of pins pass through the openings to contact the conductive contacts on the control motherboard.

Another aspect of the disclosure provides the heating member includes of one of a wrapped cotton fabric heating element and a microporous ceramic heating element.

Another aspect of the disclosure includes the base further having a plurality of ventilation holes in a circumferential direction of a bottom surface of the base; wherein an air intake hole connected to the air intake chamber; and wherein vent holes are provided at the bottom surface of the containment receptacle.

Another aspect of the disclosure includes the essential oil storage cartridge having an essential oil filling hole; and wherein the essential oil storage chamber includes a plurality of air outlet holes provided on a top surface of the essential oil storage chamber.

Another aspect of the disclosure includes aroma diffuser further having a top cover movably connected to the base through a hinge, wherein the top cover can be set on the base and a bottom portion of a wall of one side of the top cover is concave inward to form a buckle catch mechanism with a corresponding top portion of a wall of one side of the base.

Another aspect of the disclosure includes a top portion of the essential oil storage cartridge protrudes outside of the containment receptacle, and a bottom portion of the top cover is concave upwards to form an upper inset for accommodating the top portion of the essential oil storage cartridge protruding outside of the containment receptacle.

Another aspect of the disclosure includes the top cover having a fan blowing from bottom to top, the upper accommodating groove is provided with a plurality of through holes, a top surface of the top cover is provided with a plurality of aromatherapy ejection holes connected to a position of the plurality of through holes, and the fan is placed between the plurality of aromatherapy ejection holes and the position of the plurality of through holes.

Another aspect of the disclosure includes a battery electrically connected to the control motherboard is also arranged in the base; wherein a battery interface for charging the battery is provided on a side of the base; wherein a timer is also integrated on the control motherboard; and wherein a signal light and an operation button are also provided on a top surface of the base.

To the accomplishment of the foregoing and related ends, the one or more aspects include the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

These and other aspects of the invention will become more fully understood upon a review of the detailed description, which follows.

### BRIEF DESCRIPTION OF THE DRAWINGS

So that the manner in which the above-recited features of the present disclosure can be understood in more detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 is a top-down perspective view of an aroma diffuser of the present disclosure;

FIG. 2 is a top-down cross-sectional perspective view of the aroma diffuser of FIG. 1 taken along A-A;

FIG. 3 is a bottom-up exploded perspective view of the aroma diffuser of FIG. 1; and, FIG. 4 is a top-down perspective view of a base and an essential oil storage container of the aroma diffuser of FIG. 1.

FIG. 5 is an exploded perspective view of an essential oil storage container with a base and a cover of the aroma diffuser of FIG. 1 configured in accordance with one aspect of the present disclosure.

FIG. 6 is a bottom plan view of the base of the essential oil storage container of the aroma diffuser of FIG. 5.

FIG. 7 is a top plan view of the base of the essential oil storage container of the aroma diffuser of FIG. 5.

To facilitate understanding, identical reference numerals have been used, where possible, to designate identical elements that are common to the figures. In addition, certain elements described here may not be illustrated to avoid cluttering of the figures to allow easier review thereof. It is contemplated that elements disclosed in one aspect may be beneficially used in other aspects without specific recitation.

### DETAILED DESCRIPTION

Various aspects of an aroma diffuser are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different aroma diffusers, some of which are illustrated by way of example in the figures and in the following description of the preferred aspects. The detailed description and drawings are merely illustrative of the disclosure rather than limiting, the scope of the disclosure being defined by the appended claims and equivalents thereof.

It should be noted that when a component is expressed as “connected” or “coupled” to another component, it may refer to that component being directly connected/coupled to the other component or there may be one or more other components in between. In addition, the terms “upper”, “lower”, “left”, “right”, “top”, and “bottom” relate to the orientation or positional relationship indicated based on the orientation or positional relationship shown in the drawings, and should be understood that they are for the convenience of describing various aspects of the present disclosure and simplifying the description, and does not indicate nor imply that the device or element referred to must have a specific orientation, be constructed and operated in a specific orientation, and therefore should not be construed as a limitation on the present disclosure. Moreover, the terms “first”, “second”, etc., are used for descriptive purposes only and should not be construed as indicating or implying relative importance. Furthermore, any terms for motion or direction used herein, such as “move”, “slide”, “press”, “in”, “out”, “clockwise”, “counterclockwise”, etc., are provided as examples of possible movement or rotation in the described examples and should not be limiting unless otherwise used in the claims.

Unless otherwise defined, all technical and scientific terms used in this specification have the same meanings as generally understood by those skilled in the art of the present disclosure. The terms used in the description of the present disclosure are for the purpose of describing specific embodiments only and are not intended to limit the invention.

To make the purpose, technical scheme and advantages of the present disclosure clearer, the following is a further detailed explanation of the aroma diffuser in combination with the attached drawings and described embodiments. It should be understood that the specific embodiments described here are only used to explain the present disclosure and are not used to qualify or otherwise limit the present invention.

In accordance to one aspect of the present disclosure, an aroma diffuser having an electric heating atomizer that includes a base, an essential oil storage cartridge and an electric heating assembly, a top portion of the base has a recess for a storage space, which otherwise may be referred to as a containment receptacle, of the essential oil storage cartridge. The base also contains a control motherboard and the essential oil storage cartridge is arranged in the storage and is capable of being moved into or out of the storage. The electric heating assembly is embedded on a bottom portion of the essential oil storage cartridge, and when the essential oil storage cartridge is placed into the storage space, the electric heating assembly is electrically connected with the control motherboard. The electric heating element provides atomization of aromatherapy essential oil stored in the essential oil storage cartridge.

In one preferred aspect of the present disclosure, the essential oil storage cartridge is separated by a partition into an essential oil storage cavity and an air intake cavity interconnected at top portions thereof, and the essential oil storage cavity is filled with an absorbent material imbued with the aromatherapy oil.

In another preferred aspect of the present disclosure, the electric heating assembly includes a heating element and electrical contacts connected to the heating element. The control motherboard also includes matching electrical contacts, and the storage space of the essential oil storage cartridge is provided with openings to pass through the electrical contacts of the heating element to electrically connect to the matching electrical contacts on the control motherboard when the essential oil storage cartridge is inserted into the storage space.

In yet another preferred aspect of the present disclosure, the absorbent material is a cotton-based material, and the heating element is in contact with the absorbent material. The heating element may be wrapped in a material compatible to the absorbent material such as cotton fabric. A microporous ceramic heating element may also be used.

In still yet another preferred aspect of the present disclosure, a plurality of ventilation holes is provided in a circumferential direction of a lower side of the base, and one or more through holes are provided on a bottom surface of the storage space an air intake hole connected to the air intake cavity and ventilation hole is provided at the bottom of the accommodating groove.

In still yet another preferred aspect of the present disclosure, the top of the essential oil storage cartridge is provided with an essential oil filling hole and a plurality of air outlet holes connected to the essential oil storage cavity.

In still yet another preferred aspect of the present disclosure, the aroma diffuser also includes a top cover for the base The top cover is electrically connected to the base through a set of pins when the top cover can be set on the base, and a bottom wall on one side of the top cover is concave inward to form a tab or latch connection.

In still yet another preferred aspect of the present disclosure, the top of the essential oil storage cartridge protrudes outside the holding tank, and a bottom surface of the top cover has a concave portion to form an upper holding receptacle for accommodating the protruding portion of the essential oil storage cartridge.

In still yet another preferred aspect of the present disclosure, the top cover includes a fan blowing from bottom to top, the upper holding receptacle is provided with a plurality of holes. In addition, a top portion of the top cover is provided with a plurality of aromatherapy nozzle holes and the fan is arranged between the aromatherapy nozzle hole and the plurality of holes.

In still yet another preferred aspect of the present disclosure, the base also includes a battery electrically connected to the control motherboard, and a side surface of the base is provided with a battery interface for charging the battery. The control motherboard is also integrated with a timer, and the top surface of the base is also provided with a signal light and an operation button.

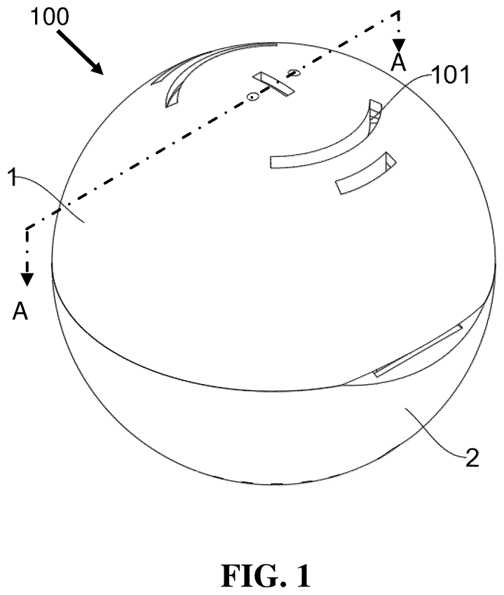

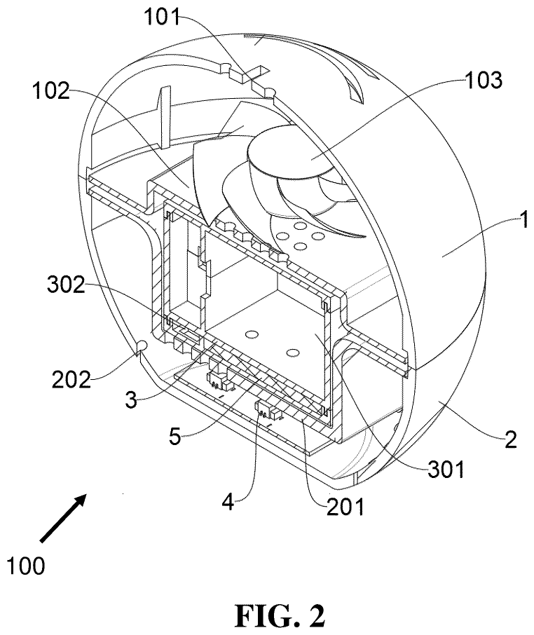

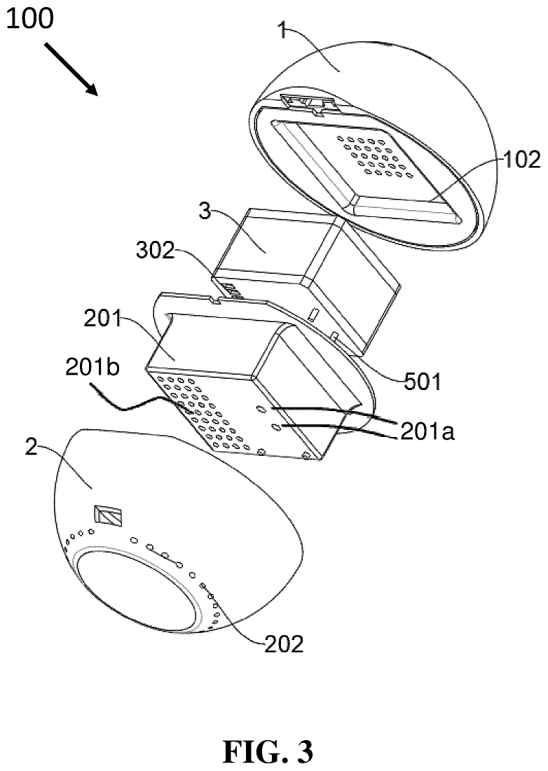

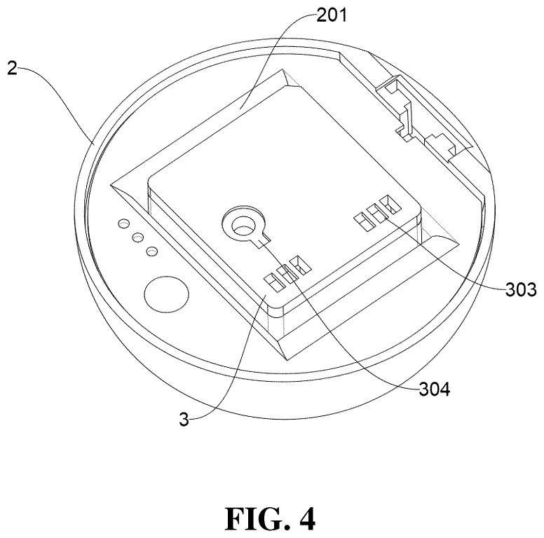

FIGS. 1-4 illustrates an aroma diffuser 100 that includes a top cover 1 and a base 2. In one aspect of the present disclosure, the base 2 is configured to receive an essential oil storage cartridge 3 with an integrated electric heating assembly, as further detailed herein. Referring to FIG. 2, a top portion of the base 2 includes an opening to hold a containment receptacle 201, and the base 2 also contains a control motherboard 4. Also referring to FIG. 3 while still referring to FIG. 2, the essential oil storage cartridge 3 may be arranged in the containment receptacle 201 such that the essential oil storage cartridge 3 may be inserted into or removed from the containment receptacle 201. The essential oil storage cartridge 3 includes an electric heating assembly embedded at a bottom portion thereof, and when the essential oil storage cartridge 3 is inserted into the containment receptacle 201, the electric heating assembly is electrically connected with the control motherboard 4. The control motherboard 4 controls the electric heating assembly so that the electric heating assembly heats up and atomizes any essential oil contained in the essential oil storage cartridge 3. The essential oil storage cartridge 3 preferably includes an electronic device, such as an integrated circuit (IC), to which the control motherboard 4 may interface, as further described herein.

In one aspect of the present disclosure, the top cover 1 is configured such that a bottom portion is concaved inward to form one part of a cavity that covers the essential oil storage cartridge 3. The top cover 1 may also include a tab that is matched to an insert on the base 2 to mechanically secure the top cover to the base 2. A snap-fit structure may also be used to mechanically secure the top cover 1 and the base 2. The top cover 1 may be opened upwards so that the essential oil storage cartridge 3 can be replaced. The essential oil storage cartridge 3 can be covered through the top cover 1 to limit the installation position of the essential oil storage cartridge 3. As further detailed herein, the top cover 1 is electrically connected to the base 2 once the top cover 1 is placed on the base 2.

In one aspect of the present disclosure, the electric heating assembly is pre-installed and embedded in the essential oil storage cartridge 3 to be in thermal contact with the essential oil contained therein. The essential oil storage cartridge 3 can be removed as a complete unit when it is desirable to use the aroma diffuser 100 with a new essential oil that may have a scent different from the essential oil contained in the essential oil storage cartridge 3. Specifically, the essential oil storage cartridge 3 is first removed before a new essential oil storage cartridge is then placed in the containment receptacle 201. Similar to how the electric heating assembly of the essential oil storage cartridge 3 is electrically connected to the control motherboard 4 when the essential oil storage cartridge 3 is inserted into the containment receptacle 201, the new essential oil storage cartridge includes an associated electric heating assembly embedded therein that is also electrically connected with the control motherboard 4 when the new essential oil storage cartridge is inserted into the storage tank 201. The new essential oil storage cartridge further includes an IC that, as described herein, may have information regarding the essential oil contained herein as well as the electric heating assembly. The use of an IC customized to each specific essential oil storage cartridge provides for optimal operation of the aroma diffuser 100 regardless of what type of essential oil is contained in an inserted essential oil storage cartridge. Once connected, the new essential oil in the new essential oil storage container can be atomized effectively by the control motherboard 4 by the associated electric heating assembly.

The essential oil in the essential oil storage cartridge 3 can be quickly replaced with essential oils of different odors while avoiding fragrance mixing caused by change of aromatherapy essential oils. In addition, instead of re-filling the essential oil in the essential oil storage cartridge 3, another essential oil storage container having a full amount of essential oil with a desired scent (which may be the same or different scent) may be used. Moreover, use of essential oil storage containers having aspects of the essential oil storage cartridge 3 described herein allows for changing of essential oils atomized by the aroma diffuser 100 quickly and easily while avoiding spilling of essential oils. As noted above, mixing and cross-contamination between essential oils with different odors may be avoided because they are separately contained.

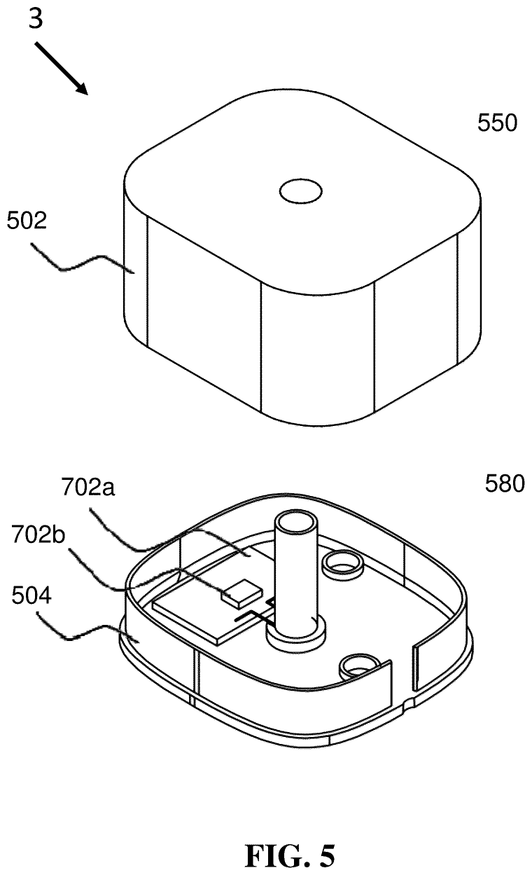

Referring to FIG. 5, in one aspect of the disclosure, the essential oil storage cartridge 3 includes a top cover 502 that has an outlet 550 for the essential oil to exit when heated. The essential oil cartridge 3 also has a base 504 that includes an IC 702b that is customized to optimize the aromatization of the specific type of essential oil contained in the essential oil storage cartridge 3. By way of example and not limitation, the IC 702b includes information such as optimal heating temperatures, length of heating for the essential oil to achieve atomization, and even safety factors such as the maximum heating temperatures for the essential oil. In addition, the IC 702b can also include information about the heating element 5, such as whether it is a fabric-wrapped or ceramic type of heating element. The control motherboard 4 may read the information from the IC 702b to optimize control of the heating element 5.

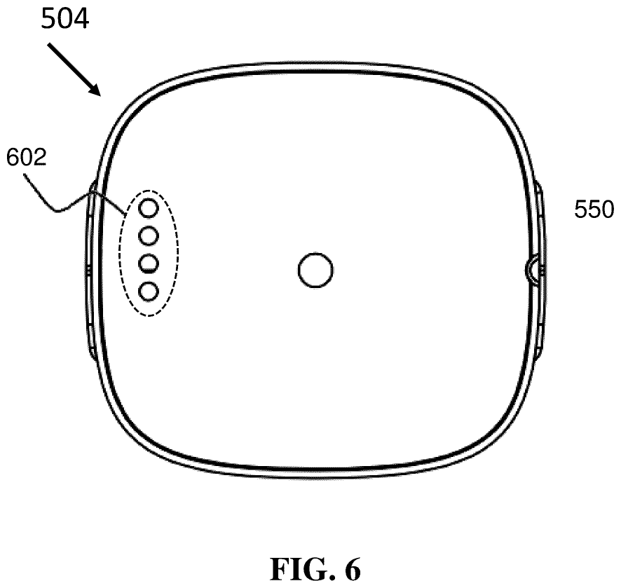

The IC 702b may be implemented as a non-volatile memory such as a read-only memory (ROM), including programmable ROM (PROM), erasable PROM (EPROM), electrically erasable PROM (EEPROM), or any similar technology that allows storage of information regarding the essential oil storage cartridge 3. In another aspect of the disclosure, the IC 702b may be implemented using electrical components such as resistors, capacitors, inductors, etc., in a circuit providing particular electrical values that, when detected by the control motherboard 4, will provides the control motherboard 4 with information about the essential oil storage cartridge 3, including how the essential oil in the essential oil storage cartridge 3 should be heated. As an example but not limitation, the IC 702b may provide a certain resistance value that is associated with a particular type of essential oil so that each specific essential oil would be assigned a different resistance value so the control motherboard 4 may handle them appropriately. The IC 702b is mounted on a PC board 702a that includes a plurality of contacts 602 to connect to the control motherboard 4 as described with reference to FIG. 6 herein.

As illustrated in FIG. 6, the plurality of contacts 602 for the essential oil storage cartridge 3 is used to couple the heating element 5 and the IC 702b to the control motherboard 4 (contacts on the control motherboard 4 are not shown to not overly complicate the figures). Although FIG. 6 shows that there are four (4) contacts in the plurality of contacts 602, there may be more or fewer contacts depending on how power/information is communicated between the PC board 702a of the essential oil storage cartridge 3 and the control motherboard 4. For example, the control motherboard 4 may use two (2) contacts of the plurality of contacts 602 as control for the heating element 5 and another two (2) contacts of the plurality of contacts 602 to communicate with the IC 702b in one aspect of the present disclosure. However, if the PC board 702a and the control motherboard 4 uses a more sophisticated communications system such as Inter-Integrated Circuit (I2C), Serial Peripheral Interface (SPI), or other communications interface, a different number of contacts may be used.

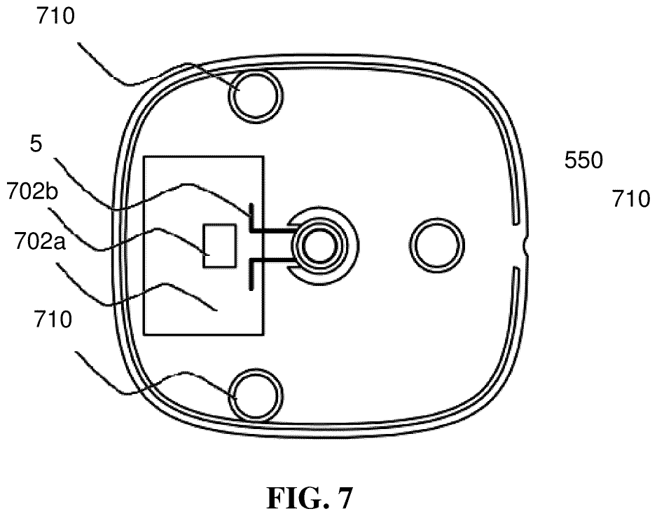

FIG. 7 illustrates a plurality of magnetic elements (magnets) 710 that assists in aligning the essential oil storage cartridge 3 to the base 2 when the essential oil storage cartridge 3 is placed in the base 2 of the aroma diffuser 100. Specifically, the plurality of magnetic elements 710 in the bottom of the essential oil storage cartridge 3 corresponds to a set of matching magnetic elements in the base 2 (not shown) of the aroma diffuser 100 so that when a user places the base 504 of the essential oil storage cartridge 3 into the base 2, the plurality of contacts 602 of the essential oil storage cartridge 3 will automatically align with contacts of the control motherboard 4. In another aspect of the disclosure, the base 2 does not use the set of matched magnetic elements in the base 2, but in place of that a set of metal elements are used to assist in aligning the plurality of contacts 602 of the essential oil storage cartridge 3.

Referring again to FIG. 2 and FIG. 3, in one aspect of the present disclosure the essential oil storage cartridge 3 is separated by a partition to form an essential oil storage chamber 301 and an air intake chamber 302, and the essential oil storage chamber 301 is filled with an absorbent material (not shown). After an essential oil is injected into the absorbent material, an aromatherapy essential oil storage container will be formed that will not leak and the essential oil will not flow quickly. Thus, problems such as liquid leakage due to the essential oil storage cartridge 3 being turned over will be eliminated. Although the absorbent material is preferably made of cotton, other absorbent materials may be used, including both natural and synthetic materials.

The electric heating assembly includes a heating member 5 and a set of pins 501 that is electrically connected to the heating member 5 that is used to connect to conductive contacts that is integrated on the control motherboard 4. The set of pins 501 is preferably made from a conductive material such as copper, but the set of pins 501 may also be created from other materials that can conduct electricity, such as any other metal. Thus, the choice of conductive materials used for the set of pins 501 should not be limiting. A set of through holes 201a is provided on a bottom portion of the containment receptacle 201 to allow the passage of the set of pins 501 to connect to the conductive contact on the control motherboard 4 when the essential oil storage cartridge 3 is inserted into the containment receptacle 201. Because the heating member 5 is in contact with the absorbent material in the essential oil storage chamber 301, the heating member 5 may be used to atomization of the essential oils infused in the absorbent material and the fragrance from the essential oil will not change after atomization by heating. The aroma diffuser 100 may be used in environments with different temperatures to quickly atomize the essential oil into the air so that the space is quickly filled with fragrance, and the heating member 5 is used as an atomizer to prevent the efficiency from being affected by the change of ambient temperature. The aroma diffuser 100 will not produce any high-frequency sounds or vibration and may be placed at will because no water is needed during operation.

In one aspect of the present disclosure, the heating member 5 may be implemented as a fabric-wrapped heating element or a microporous ceramic heating element. The heating member 5 may also include a thermistor to allow detection of temperature using circuitry in the control motherboard 4. The heating element 5 may be created using an electric heating wire and, when the heating member 5 is the fabric-wrapped heating element, cotton may be used to wrap the heating element. When the heating member 5 is the microporous ceramic heating element, the electric heating wire part is embedded in the body of the microporous ceramic heating element. The electric heating wire is preferably an electric heating wire made from platinum group metals.

In one aspect of the present disclosure, the essential oil storage cartridge 3 includes a top portion that protrudes above the containment receptacle 201, and a bottom portion of the top cover 1 includes an upper cover 102 with a shape for accommodating the top portion of the essential oil storage cartridge 3. Because the top portion of the essential oil storage cartridge 3 protrudes outside the containment receptacle 201, it is convenient for the user to remove the essential oil storage cartridge 3 from the containment receptacle 201.

Referring to FIG. 3, in one aspect of the present disclosure, air flow needed for the aroma diffuser 100 is provided through a series of openings, or holes, located on various parts of the aroma diffuser 100. In the illustrated example, the base 2 includes a plurality of air intake holes 202 in a lower surface of the base 2 that serves to allow air from the surrounding environment to be drawn into the aroma diffuser 100. Specifically, the plurality of air intake holes 202 allows air flow from a plurality of intake openings 201b at a bottom surface of the containment receptacle 201. The air flow then passes through the air intake chamber 302 to enter the essential oil storage chamber 301.

Now referring to FIG. 4, the essential oil storage cartridge 3 includes an essential oil filling hole 304 to allow essential oil to be refilled to the essential oil storage chamber 301. In addition, the essential oil storage cartridge 3 includes a plurality of outlet holes 303 coupled to the essential oil storage chamber 301 on a top surface of the essential oil storage cartridge 3. The plurality of outlet holes 303 allows air with aroma from the essential oil storage cartridge 3 to exit from the essential oil storage chamber 301.

In one aspect of the present disclosure, to disperse the scent from essential oils from the essential oil storage cartridge 3 of the aroma diffuser 100 for aromatherapy, a plurality of aromatherapy holes 101 is provided on the top cover 1, and the plurality of air intake holes 202 is designed to provide proper air flow from the bottom to the top of the aroma diffuser 100 to allow passing of environmental air to the air after mixing with atomized essential oils that can be discharged through the aromatherapy holes 101.

Referring again to FIG. 2, the top cover 1 is equipped with a fan 103 that blows bottom-up (pulling air from the plurality of air intake holes 202). In one aspect of the present disclosure, the fan 103 is placed between the plurality of aromatherapy holes 101 and the plurality of air intake holes 202. The addition of the fan 103 can assist the aromatherapy atomized gas to be dissipated more quickly.

As described herein, the top cover 1 is electrically connected to the base 2. In accordance with one aspect of the present disclosure, the base 2 accommodates a battery (not shown) connected to the control motherboard 4 so that the aroma diffuser 100 may operate without being connected to an external power source. The control motherboard 4 also includes a battery interface for charging the battery that is accessible from a side of the base 2. In addition, a timer may be integrated into the control motherboard 4 to allow the aroma diffuser 100 to automatically operate at specific times. Specifically, an operation button on top of the cover 1 is connected to the control motherboard 4 to operate the timer to control the heating working interval of the electric heating element, which can work at different intervals, so that even a small amount of essential oil can be used for a long time, and the environment is full of fragrance. In addition, the aroma diffuser 100 may include a signal light to show the state of the aroma diffuser 100.

Using the electric heating assembly in the essential oil storage cartridge, the aroma of the aromatherapy essential oil will not change after heating atomization, and can be quickly atomized into the air, so that the space is quickly filled with fragrance. The electric heating assembly is pre-installed in the essential oil storage cartridge and, as a whole, can be replaced with the replacement of the essential oil storage cartridge, so that the smell can be quickly replaced, and the problem of odor mixing will not occur. In addition, magnets placed on the bottom of the essential oil storage cartridge as well as Further, the use of the IC with information regarding the essential oil in the essential oil storage cartridge provides more concise control of the amount of heat applied to the essential oil from the heating element with no/minimal input needed from the user, thereby simplifying the operation of the aroma diffuser.

It should be noted that the above embodiments are only used to illustrate the technical solutions of the present disclosure, not to limit them; under the idea of the present disclosure, the technical features in the above embodiments or different embodiments may also be combined, the steps may be realized in any order, and there are many other variations of different aspects of the present disclosure as above, and they are not provided in detail for the sake of brevity; although the present disclosure is said in detail with reference to the foregoing embodiments, a person skilled in the art should understand that it may still modify the technical solutions said in the foregoing embodiments, or replace some of the technical features therein; and these modifications or substitutions do not make the essence of the corresponding technical solutions out of the scope of the technical solutions of the various embodiments of the present disclosure.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). As used herein, including in the claims, the term “and/or,” when used in a list of two or more items, means that any one of the listed items may be employed by itself, or any combination of two or more of the listed items may be employed. For example, if a composition is described as containing components A, B, and/or C, the composition may contain A alone; B alone; C alone; A and B in combination; A and C in combination; B and C in combination; or A, B, and C in combination.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein but is to be accorded the full scope consistent with the language claims, wherein reference to an element in the singular is not intended to mean “one and only one” unless specifically so stated, but rather “one or more.” For example, the articles “a” and “an” as used in this disclosure and the appended claims should generally be construed to mean “one or more” unless specified otherwise or clear from the context to be directed to a singular form. Unless specifically stated otherwise, the term “some” refers to one or more. Moreover, the term “or” is intended to mean an inclusive “or” rather than an exclusive “or.” That is, unless specified otherwise, or clear from the context, the phrase, for example, “X employs A or B” is intended to mean any of the natural inclusive permutations. That is, for example the phrase “X employs A or B” is satisfied by any of the following instances: X employs A; X employs B; or X employs both A and B. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

It is to be understood that the claims are not limited to the precise configuration and components illustrated above. Various modifications, changes and variations may be made in the arrangement, operation and details of the methods and apparatus described above without departing from the scope of the claims.

What is claimed is:

1. An aroma diffuser, comprising:
a base comprising:
a cavity,
a containment receptacle contained in the cavity;
a control motherboard is arranged inside the cavity of the base below the containment receptacle;
an essential oil storage cartridge comprising:
a partition separating a chamber in the essential oil storage cartridge into:
an air intake chamber; and
an essential oil storage chamber storing essential oil;
an electric heating assembly that is in thermal connectivity with the essential oil storage chamber; and
a plurality of contacts, mounted on an exterior surface of the essential oil storage cartridge, coupled to the electric heating assembly for electrically connecting the electric heating assembly to the control motherboard;
wherein the essential oil storage cartridge is removably inserted into the containment receptacle of the base.

2. The aroma diffuser of claim 1, wherein the essential oil storage cartridge further comprises an integrated circuit including information about the essential oil in the essential oil storage chamber; wherein the integrated circuit is coupled to the plurality of contacts of the essential oil storage cartridge; and wherein the control motherboard can determine how to heat the essential oil using the information from the integrated circuit.

3. The aroma diffuser of claim 2, wherein the information contained in the integrated circuit for the essential oil further comprises information regarding the electric heating assembly in the essential oil storage cartridge.

4. The aroma diffuser of claim 1, wherein the electric heating assembly is embedded into a bottom of the essential oil storage cartridge, and when the essential oil storage cartridge is inserted into the containment receptacle of the base, the electric heating assembly is electrically connected to the control motherboard to allow the control motherboard to control the electric heating assembly so as to heat and atomize the essential oil contained in the essential oil storage chamber.

5. The aroma diffuser of claim 1, wherein the partition allows the essential oil storage chamber and the air intake chamber to be connected at a top portion of the partition, and the essential oil storage chamber is filled with an absorbent material infused with the essential oil.

6. The aroma diffuser of claim 1, wherein the electric heating assembly comprises a heating member and a plurality of pins electrically connected to the heating member; wherein the control motherboard comprises conductive contacts; and wherein the containment receptacle is provided with openings on a bottom surface such that when the essential oil storage cartridge is inserted into the containment receptacle, the plurality of pins pass through the openings to contact the conductive contacts on the control motherboard.

7. The aroma diffuser of claim 6, wherein the heating member is comprised of one of a wrapped cotton fabric heating element and a microporous ceramic heating element.

8. The aroma diffuser of claim 6, wherein the base further comprises a plurality of ventilation holes in a circumferential direction of a bottom surface of the base; wherein an air intake hole connected to the air intake chamber; and wherein vent holes are provided at the bottom surface of the containment receptacle.

9. The aroma diffuser of claim 1, wherein the essential oil storage cartridge comprises an essential oil filling hole; and wherein the essential oil storage chamber comprises a plurality of air outlet holes provided on a top surface of the essential oil storage chamber.

10. The aroma diffuser of claim 1, further comprising a top cover movably connected to the base through a hinge, wherein the top cover can be set on the base and a bottom portion of a wall of one side of the top cover is concave inward to form a buckle catch mechanism with a corresponding top portion of a wall of one side of the base.

11. The aroma diffuser of claim 10, wherein a top portion of the essential oil storage cartridge protrudes outside of the containment receptacle, and a bottom portion of the top cover is concave upwards to form an upper inset for accommodating the top portion of the essential oil storage cartridge protruding outside of the containment receptacle.

12. The aroma diffuser of claim 10, wherein the top cover comprises a fan blowing from bottom to top, the upper accommodating groove is provided with a plurality of through holes, a top surface of the top cover is provided with a plurality of aromatherapy ejection holes connected to a position of the plurality of through holes, and the fan is placed between the plurality of aromatherapy ejection holes and the position of the plurality of through holes.

13. The aroma diffuser of claim 1, wherein a battery electrically connected to the control motherboard is also arranged in the base; wherein a battery interface for charging the battery is provided on a side of the base; wherein a timer is also integrated on the control motherboard; and wherein a signal light and an operation button are also provided on a top surface of the base.

14. The aroma diffuser of claim 1, wherein the essential oil storage cartridge further comprises an arrangement of magnets on the exterior surface of the essential oil storage cartridge that aligns the plurality of contacts of the essential oil storage cartridge to electrically connect to a matching set of contacts on the control motherboard when the essential oil storage cartridge is inserted into the containment receptacle of the base.
